(19) United States
(12) Patent Application Publication
(10) Pub. No.: US 2023/0064186 A1
(43) Pub. Date: Mar. 2, 2023

(54) MICROORGANISM COLLECTION DEVICE

(71) Applicant: Parasol Medical, LLC, Buffalo Grove, IL (US)

(72) Inventors: Jacob D. Stephens, Lowell, MI (US); Michael D. Kilcran, Antioch, IL (US); Lucas W. Stephens, Lowell, MI (US)

(73) Assignee: Parasol Medical, LLC, Buffalo Grove, IL (US)

(21) Appl. No.: 17/898,377

(22) Filed: Aug. 29, 2022

Related U.S. Application Data
(60) Provisional application No. 63/238,741, filed on Aug. 30, 2021.

(51) Int. Cl. C12M 1/28; B01L 3/00; C12M 1/26

(52) U.S. Cl. CPC C12M 1/28; B01L 3/502; C12M 33/22

(57) ABSTRACT

A microorganism collection device comprises: (a) a base, the base comprising (i) a reservoir comprising a bottom and a side wall extending up from the bottom, (ii) a culture medium within the reservoir, the culture medium disposed upon the bottom of the reservoir and comprising a top surface that is elevated from the bottom higher than the side wall of the reservoir, (iii) a first tab extending laterally outward relative to the reservoir; and (b) a lid comprising (i) an associated state where the lid is covering the culture medium and (ii) a dissociated state where the lid is separated from the base and not covering the culture medium.

### CROSS-REFERENCE TO RELATED APPLICATION

This application claims priority to and the benefit under 35 U.S.C. § 119(e) of U.S. Provisional Patent Application No. 63/238,741, filed on 30 Aug. 2021, entitled MICROORGANISM COLLECTION DEVICE, the disclosure of which is hereby incorporated herein by reference in its entirety.

### BACKGROUND

There are a variety of devices that include a culture medium to test for the presence of microorganisms. For example, a dip slide includes a culture medium that can be dipped into a liquid. Microorganisms, if present in the liquid, transfer from the liquid to the culture medium, which can then be incubated. In addition, an agar plate is often used to culture microorganisms. An agar plate is typically a round Petri dish that contains culture medium. An inoculator may be used to transfer the sample to be tested to the culture medium in the agar plate.

However, there is a problem in that both the dip slide and the agar plate are difficult to handle when collecting a sample.

### SUMMARY

The present disclosure addresses that problem with a microorganism collection device with a base that has a generally rectangular outline and one or more outwardly extending tabs that facilitate handling of the microorganism collection device.

According to a first aspect of the present disclosure, a microorganism collection device comprises: (a) a base, the base comprising (i) a reservoir comprising a bottom and a side wall extending up from the bottom, (ii) a culture medium within the reservoir, the culture medium disposed upon the bottom of the reservoir and comprising a top surface that is elevated from the bottom higher than the side wall of the reservoir, (iii) a first tab extending laterally outward relative to the reservoir; and (b) a lid comprising (i) an associated state where the lid is covering the culture medium and (ii) a dissociated state where the lid is separated from the base and not covering the culture medium.

According to a second aspect of the present disclosure, the microorganism collection device of the first aspect, wherein the side wall of the reservoir comprises (i) a first pair of opposing side wall sections and (ii) a second pair of opposing side wall sections that are generally orthogonal to the first pair of opposing side wall sections.

According to a third aspect of the present disclosure, the microorganism collection device of any one of the first through second aspects, wherein the top surface of the culture medium is convex.

According to a fourth aspect of the present disclosure, the microorganism collection device of any one of the first through third aspects, wherein (i) the reservoir further comprises dividers extending upward from the bottom and disposed below the culture medium; and (ii) the dividers are visible through the culture medium.

According to a fifth aspect of the present disclosure, the microorganism collection device of any one of the first through fourth aspects, wherein the base further comprises a lid reception section surrounding the reservoir, the lid reception section comprising a bottom and a side wall extending up from the bottom, the side wall of the lid reception section extending around the side wall of the reservoir.

According to a sixth aspect of the present disclosure, the microorganism collection device of the fifth aspect, wherein the side wall of the lid reception section of the base comprises (i) a first pair of opposing side wall sections and (ii) a second pair of opposing side wall sections that are generally orthogonal to the first pair of opposing side wall sections.

According to a seventh aspect of the present disclosure, the microorganism collection device of any one of the first through sixth aspects, wherein (i) the lid further comprises a top and a side wall extending down from the top; and (ii) when the lid is in the associated state, the top of the lid is disposed over the culture medium, and the side wall of the lid extends around the side wall of the reservoir.

According to an eighth aspect of the present disclosure, the microorganism collection device of the seventh aspect, wherein (i) the lid further comprises a projection projecting upward from the top of the lid; and (ii) the base further comprises a recessed portion at an underside of the base, the recessed portion of the microorganism collection device configured to receive the projection of the lid of another microorganism collection device to allow stacking of the microorganism collection devices.

According to a ninth aspect of the present disclosure, the microorganism collection device of the fifth aspect, wherein (i) the side wall of the lid comprises a bottom edge and, when the lid is in the associated state, the bottom edge of the lid faces the bottom of the lid reception section; and (ii) when the lid is in the associated state, the side wall of the lid reception section of the base extends around the side wall of the lid, and the side wall of the lid is disposed between the side wall of the lid reception section and the side wall of the reservoir of the base.

According to a tenth aspect of the present disclosure, the microorganism collection device of any one of the first through ninth aspects, wherein (i) the lid further comprises an unlocked state and a first locked state; (ii) the lid is movable to, from, and between the unlocked state and the first locked state; and (iii) when moving to, from, and between the unlocked state and the first locked state, the bottom edge of the lid slides laterally relative to the base.

According to an eleventh aspect of the present disclosure, the microorganism collection device of the tenth aspect, wherein (i) the lid further comprises a second locked state and a slot; (ii) when the lid is in the first locked state, air is able to flow from an environment exterior to the microorganism collection device, through the slot of the lid, and to the culture medium; and (iii) the base further comprises a projection that cooperates with the slot when the lid is in the second locked state to reduce or prevent air from flowing from the environment exterior to the microorganism collection device, through the slot of the lid, and to the culture medium.

According to a twelfth aspect of the present disclosure, the microorganism collection device of any one of the first through eleventh aspects, wherein (i) the lid further comprises an outwardly extending projection, the outwardly extending projection comprising a lateral length; and (ii) the base further comprises a receiver for the outwardly extending projection of the lid to releasably attach the lid to the base, the receiver comprising a lateral length that is equal to or longer than the lateral length of the outwardly extending projection of the lid.

According to a thirteenth aspect of the present disclosure, the microorganism collection device of the eleventh aspect, wherein (i) the lid further comprises an outwardly extending projection, the outwardly extending projection comprising a lateral length; (ii) the base further comprises a receiver for the outwardly extending projection of the lid to releasably attach the lid to the base, the receiver comprising a lateral length that is equal to or longer than the lateral length of the outwardly extending projection of the lid; (iii) the receiver of the base further comprises a first overhang and a second overhang; (iv) the first overhang extends over the outwardly extending projection of the side wall of the lid when the lid is in the first locked state but not when the lid is in the unlocked state or the second locked state; and (v) the second overhang extends over the outwardly extending projection of the side wall of the lid when the lid is in the second locked state but not when the lid is in the unlocked state or the first locked state.

According to a fourteenth aspect of the present disclosure, the microorganism collection device of any one of the first through thirteenth aspects, wherein (i) the first tab comprises a top that has an angular relationship with the bottom of the reservoir; and (ii) the first tab is movable to, from, and between a first position and a second position where the angular relationship between the top of the first tab and the bottom of the reservoir is greater than in the first position.

According to a fifteenth aspect of the present disclosure, the microorganism collection device of the fourteenth aspect, wherein the first tab of the base moves relative to the reservoir about a hinge when moving to, from, and between the first position and the second position.

According to a sixteenth aspect of the present disclosure, the microorganism collection device of the fifteenth aspect, further comprising: a second tab extending outward relative to the reservoir away from the first tab; wherein (i) the second tab comprises a top that has an angular relationship with the bottom of the reservoir; (ii) the second tab is movable to, from, and between a first position, and a second position where the angular relationship between the top of the second tab and the bottom of the reservoir is greater than in the first position; and (iii) the second tab of the base moves relative to the reservoir about a hinge when moving to, from, and between the first position and the second position.

According to a seventeenth aspect of the present disclosure, the microorganism collection device of any one of the first through sixteenth aspects further comprises a machine readable indicia.

According to an eighteenth aspect of the present disclosure, a microorganism collection device comprises: (a) a base, the base comprising (i) a reservoir comprising a bottom and a side wall extending up from the bottom, and (ii) a culture medium within the reservoir, the culture medium disposed upon the bottom of the reservoir and comprising a top surface that is elevated from the bottom higher than the side wall of the reservoir; and (b) a lid comprising (i) a top, (ii) a side wall extending down from the top, the side wall comprising a bottom edge, (iii) an associated state where the lid is coupled to the base and the top of the lid is disposed over the culture medium, (iv) a dissociated state where the lid is not coupled to the base and not covering the culture medium, (v) an unlocked state, (vi) a first locked state, and (vii) a second locked state; wherein, the lid is movable to, from, and between the unlocked state, the first locked state, and the second locked state; wherein, when moving to, from, and between the unlocked state to the first locked state, the bottom edge of the lid slides laterally relative to the base in a first direction; and wherein, when moving to, from, and between the unlocked state to the second locked state, the bottom edge of the lid slides laterally relative to the base in a second direction, the first direction and the second direction being opposites.

According to a nineteenth aspect of the present disclosure, the microorganism collection device of the eighteenth aspect, wherein (i) the side wall of the lid further comprises an outwardly extending projection, the outwardly extending projection comprising a lateral length; (ii) the base further comprises a lid reception section around the reservoir, the lid reception section comprising a bottom and a side wall extending up from the bottom, the side wall of the lid reception section extending around the side wall of the reservoir; the side wall of the lid reception section of the base comprises a receiver configured to cooperate with the outwardly extending projection of the lid to attach the lid to the base, the receiver comprising (i) a lateral length that is longer than the lateral length of the outwardly extending projection of the lid, (ii) a first overhang, and (iii) a second overhang; the first overhang extends over the outwardly extending projection of the side wall of the lid when the lid is in the first locked state but not when the lid is in the unlocked state or the second locked state; and the second overhang extends over the outwardly extending projection of the side wall of the lid when the lid is in the second locked state but not when the lid is in the unlocked state or the first locked state.

According to a twentieth aspect of the present disclosure, a microorganism collection device comprises: (a) a base, the base comprising (i) a reservoir comprising a bottom and a side wall extending up from the bottom, and (ii) a culture medium within the reservoir, the culture medium disposed upon the bottom of the reservoir and comprising a top surface that is elevated from the bottom higher than the side wall of the reservoir; and a lid comprising (i) a top, (ii) a side wall extending down from the top, the side wall of the lid comprising a slot, (iii) an associated state where the lid is coupled to the base and the top of the lid is disposed over the culture medium, (iv) a dissociated state where the lid is not coupled to the base and not covering the culture medium, (v) an unlocked state, (vi) a first locked state in which air is able to flow from an environment exterior to the microorganism collection device, through the slot of the side wall, and to the culture medium, and (vii) a second locked state in which the slot cooperates with a projection of the base to reduce or prevent air from flowing from the environment exterior to the microorganism collection device, through the slot of the side wall, and to the culture medium; wherein, the lid is movable to, from, and between the unlocked state, the first locked state, and the second locked state.

According to a twenty-first aspect of the present disclosure, the microorganism collection device of the twentieth aspect, wherein the side wall of the reservoir comprises (i) a first pair of opposing side wall sections that are planar and (ii) a second pair of opposing side wall sections that are planar and orthogonal to the first pair of opposing side wall sections.

These and other features, advantages, and objects of the present invention will be further understood and appreciated by those skilled in the art by reference to the following specification, claims, and appended drawings.

### BRIEF DESCRIPTION OF THE DRAWINGS

FIG. 1 is a side perspective view of a microorganism collection device of the present disclosure, illustrating (i) a base with a reservoir and culture medium disposed in the reservoir, and (b) a lid in a disassociated state relative to the base;

FIG. 2 is an overhead view of the base of the microorganism collection device of FIG. 1, illustrating a first tab and a second tab extending laterally outward in opposite directions relative to the reservoir;

FIG. 3 is an elevation view of a cross-section of the base of the microorganism collection device of FIG. 1 taken through line III-III of FIG. 2, illustrating the base further including a lid reception section that surrounds the reservoir, the lid reception section and the reservoir both including a side wall;

FIG. 4 is a side perspective view of the microorganism collection device of FIG. 1, illustrating the lid in an associated state covering the culture medium of the base, the lid further including an outwardly extending projection that extends outwardly from a sidewall of the lid, and the base further including a receiver for the outwardly extending projection of the lid;

FIG. 5 is an elevation view of the microorganism collection device of FIG. 1 taken through line IV-IV of FIG. 4, illustrating the lid reception section of the lid having received the sidewall of the lid when the lid is in the associated state with the base covering the culture medium of the base, and the sidewall of the lid disposed between the sidewall of the lid reception section of the base and the sidewall of the reservoir of the base;

FIGS. 6A-6C are side elevation views of the microorganism collection device of FIG. 1 with the lid in the associated state covering the culture medium of the base, illustrating the lid in an unlocked state (FIG. 6A), the lid having moved in a second direction relative to the base to a second locked state (FIG. 6B) with a second overhang of the receiver cooperating with the outwardly extending projection of the lid to maintain the lid in the second locked state, and the lid having moved in a first direction (opposite of the second direction) relative to the base to a first locked state (FIG. 6C) with the first overhang of the receiver cooperating with the outwardly extending projection of the lid to maintain the lid in the first locked state;

FIG. 7A is a side elevation view of a cross-section of the microorganism collection device of FIG. 1 with the lid in the first locked state (FIG. 6C) taken through line VIIA-VIIA of FIG. 4, illustrating air flowing from an environment external to the microorganism collection device, through slots in the side wall of the lid, and to the culture medium;

FIG. 7B is a side elevation view of a cross-section of the microorganism collection device of FIG. 1 with the lid in the second locked state (FIG. 6B) taken through line VIIB-VIIB of FIG. 4, illustrating projections of the base filling the slots of the lid to prevent the air from flowing from the environment external to the microorganism collection device, through the slots in the side wall of the lid, and to the culture medium;

FIG. 8 is bottom perspective view of the lid of the microorganism collection device of FIG. 1, illustrating the slots at a bottom edge of the side wall of the lid through which air can flow to the culture medium when the lid is in the first locked state;

FIG. 9 is an elevation view of the base of the microorganism collection device in an upside-down orientation, illustrating the first tab and the second tab both having moved from a first position (FIG. 1) to a second position where an angular relationship of a top of the first tab and the second tab respectively relative to the reservoir of the base has changed; and

FIG. 10 is a bottom perspective view of a pair of microorganism collection devices of FIG. 1, illustrating the base of the microorganism collection device at the top having a recessed portion at an underside of the base that cooperates with a projection from a of the lid of the microorganism collection device at the bottom, thus, allowing stacking of the microorganism collection devices.

### DETAILED DESCRIPTION

For purposes of description herein, it is to be understood that the specific devices and processes illustrated in the attached drawings, and described in the following specification, are simply exemplary embodiments of the inventive concepts defined in the appended claims. Hence, physical characteristics relating to the embodiments disclosed herein are not to be considered as limiting, unless the claims expressly state otherwise.

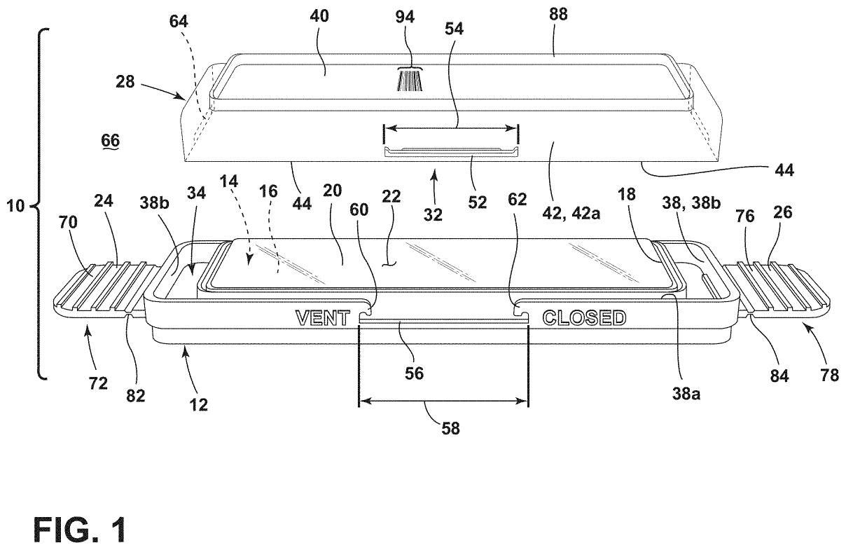

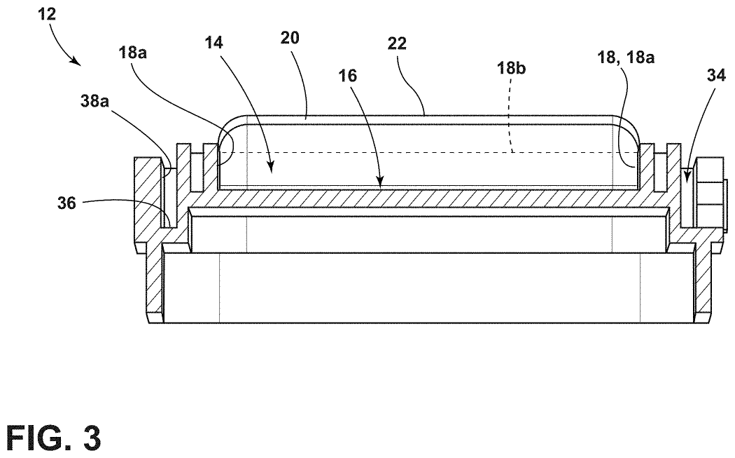

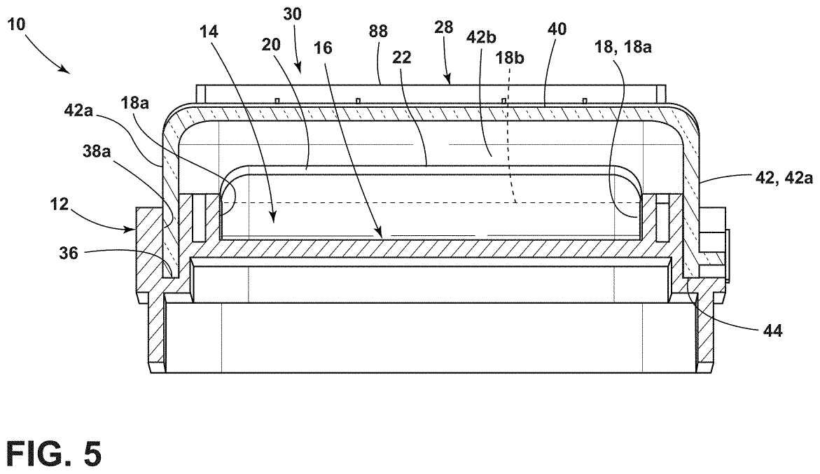

Referring now to FIGS. 1-5, a microorganism collection device 10 includes a base 12. The base 12 includes a reservoir 14. The reservoir 14 includes a bottom 16 and a side wall 18 extending up from the bottom 16.

The base further includes a culture medium 20. The culture medium 20 is disposed within the reservoir 14, upon the bottom 16 of the reservoir 14. The culture medium 20 has a top surface 22. In embodiments, the top surface 22 is elevated from the bottom 16 higher than the side wall 18 of the reservoir 14. In embodiments, the side wall 18 of the reservoir 14 includes a first pair of opposing side wall sections 18a and a second pair of opposing side wall sections 18b. The second pair of opposing side wall sections 18b are orthogonal or generally orthogonal to the first pair of opposing side wall sections 18a. The first pair of opposing side wall sections 18a can be planar. The second pair of opposing side wall sections 18b can be planar. The culture medium 20 can include agar. The culture medium 20 can be solid or semi-solid.

The microorganism collection device 10 further includes a first tab 24 and, in embodiments, a second tab 26. Both the first tab 24 and the second tab 26 extend laterally outward relative to the reservoir 14. The second tab 26 extends outward relative to the reservoir 14 away from the first tab 24.

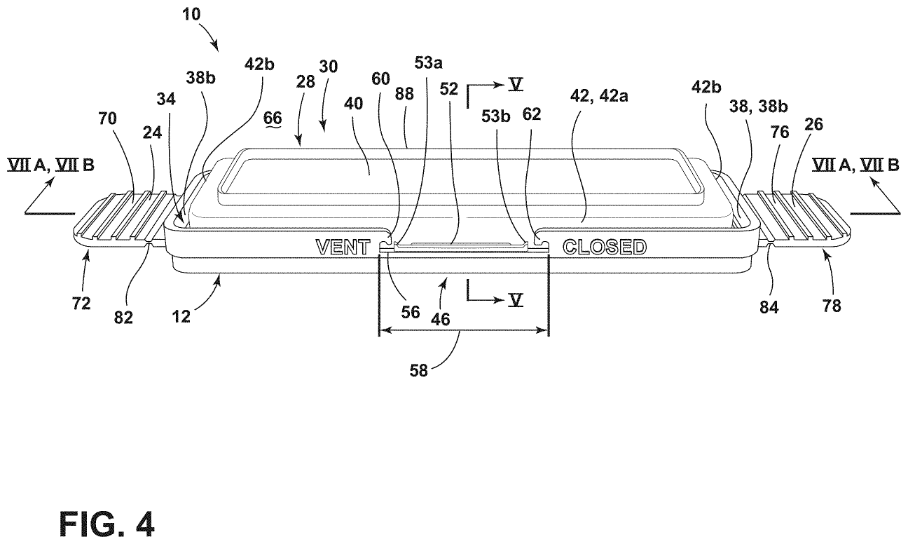

The microorganism collection device 10 further includes a lid 28. The lid 28 includes an associated state 30 (see FIG. 4) where the lid 28 is associated with (e.g., lying upon, attached to, or disposed over) the base 12 and covering the culture medium 20. In addition, the lid 28 includes a dissociated state 32 (see FIG. 1) where the lid 28 is not associated with (e.g., decoupled, not lying upon, or separated from) the base 12 and, thus, does not cover the culture medium 20. The top surface 22 of the culture medium 20 at a cross-section of the culture medium 20 can be convex.

In embodiments, the base 12 further includes a lid reception section 34. The lid reception section 34 surrounds the reservoir 14. The lid reception section 34 includes a bottom 36 and a side wall 38. The side wall 38 extends up from the bottom 36. The side wall 38 of the lid reception section 34 of the base 12 comprises a first pair of opposing side wall sections 38a and a second pair of opposing side wall sections 38b that are generally orthogonal or orthogonal to the first pair of opposing side wall sections 38a. The first pair of opposing side wall sections 38a can be planar. The second pair of opposing side wall sections 38b can be planar. The side wall 38 of the lid reception section 34 of the base 12 can (as illustrated) extend around the side wall 18 of the reservoir 14.

The lid 28 further comprises a top 40 and a side wall 42. The side wall 42 extends down from the top 40. In the associated state 30, the top 40 of the lid 28 is disposed over the culture medium 20, and the side wall 42 of the lid 28 extends around the side wall 18 of the reservoir 14. For example, in embodiments, the side wall 42 of the lid 28 includes a first pair of opposing side wall sections 42a and a second pair of opposing side wall sections 42b that are generally orthogonal or orthogonal to the first pair of opposing side wall sections 42a. The first pair of opposing side wall sections 42a can be planar. The second pair of opposing side wall sections 42b can be planar. When the lid 28 is in the associated state 30, the first pair of opposing side wall sections 42a of the lid 28 are outward of the first pair of opposing side wall sections 18a of the reservoir 14. In addition, when the lid 28 is in the associated state 30, the second pair of opposing side wall sections 42b of the lid 28 are outward of the second pair of opposing side wall sections 18b of the reservoir 14.

The side wall 42 of the lid 28 comprises a bottom edge 44. When the lid 28 is in the associated state 30, the bottom edge 44 of the lid 28 faces and, in embodiments, sits upon the bottom 36 of the lid reception section 34. When the lid 28 is In the associated state 30, the side wall 38 of the lid reception section 34 of the base 12 extends around the side wall 42 of the lid 28, and the side wall 42 of the lid 28 is at least partially disposed between the side wall 38 of the lid reception section 34 and the side wall of the reservoir 14 of the base 12. In embodiments, when the lid 28 is in the associated state 30, one of the first pair of opposing side wall sections 42a is at least partially disposed between one of the first pair of opposing side wall sections 38a of the lid reception section 34 of the base 12 and one of the first pair of opposing side wall sections 18a of the reservoir 14 of the base 12. Likewise, when the lid 28 is in the associated state 30, the other of the first pair of opposing side wall sections 42a is at least partially disposed between the other of the first pair of opposing side wall sections 38a of the lid reception section 34 of the base 12 and the other of the first pair of opposing side wall sections 18a of the reservoir 14 of the base 12. Similarly, when the lid 28 is in the associated state 30, one of the first pair of opposing side wall sections 42b is at least partially disposed between one of the first pair of opposing side wall sections 38b of the lid reception section 34 of the base 12 and one of the first pair of opposing side wall sections 18b of the reservoir 14 of the base 12. Likewise, when the lid 28 is in the associated state 30, the other of the first pair of opposing side wall sections 42b is at least partially disposed between the other of the first pair of opposing side wall sections 38b of the lid reception section 34 of the base 12 and the other of the first pair of opposing side wall sections 18b of the reservoir 14 of the base 12. Note that, in embodiments (such as that illustrated), the first pair of opposing side wall sections 42a and the second pair of opposing side wall sections 42b are elevated higher than both (i) the first pair of opposing side wall sections 18a and the second pair of opposing side wall sections 18b of the reservoir 14 of the base 12 and (ii) the first pair of opposing side wall sections 38a and the second pair of opposing side wall sections 38b of the lid reception section 34 of the base 12. The top 40 of the lid 28 thus extends over the top surface 22 of the culture medium 20. The lid 28 and the reservoir 14 of the base 12 thus at least partially encapsulate the culture medium 20.

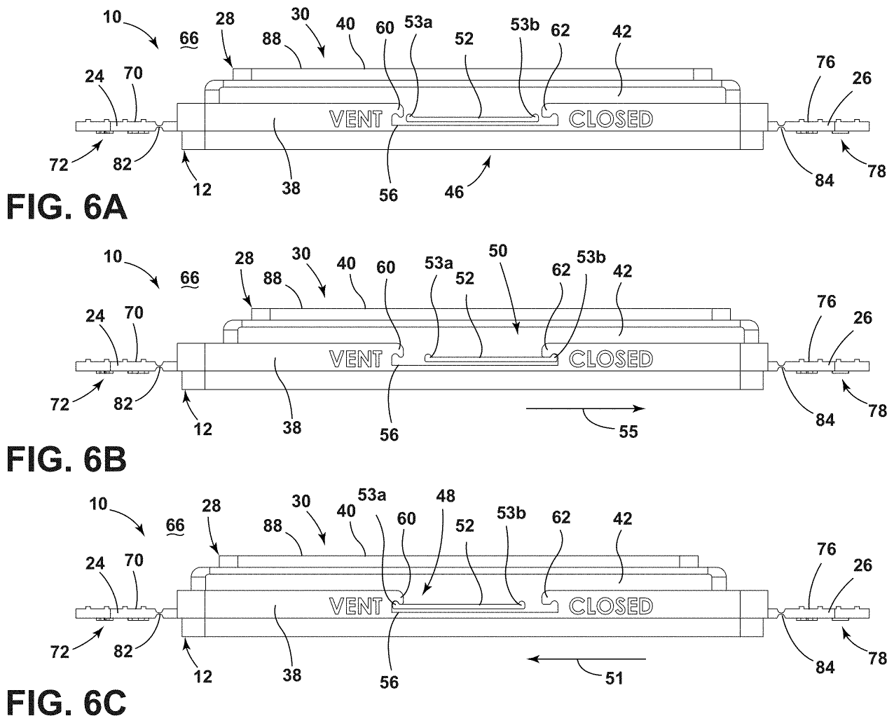

Referring now additionally to FIGS. 6A-6C, the lid 28 further comprises an unlocked state 46 (see FIG. 6A), a first locked state 48 (see FIG. 6C), and a second locked state 50 (see FIG. 6B). The lid 28 is movable to, from, and between the unlocked state 46, the first locked state 48, and the second locked state 50. When moving to, from, and between the unlocked state 46, the first locked state 48, and the second locked state 50, the bottom edge 44 of the lid 28 slides laterally relative to the bottom 36 of the lid reception section 34 of the base 12. When moving to, from, and between the unlocked state 46 to the first locked state 48, the bottom edge 44 of the lid 28 slides laterally relative to the base 12 in a first direction 51. When moving to, from, and between the unlocked state 46 to the second locked state 50, the bottom edge 44 of the lid 28 slides laterally relatively to the base 12 in a second direction 55. The first direction 51 and the second direction 55 are opposites. In embodiments, the bottom edge 44 of the lid 28 slides laterally upon the base 12 when moving to, from, and between the unlocked state 46 to the first locked state 48, and the unlocked state 46 to the second locked state 50.

The side wall 42 of the lid 28 further comprises an outwardly extending projection 52, the outwardly extending projection 52 comprising a lateral length 54. In embodiments, the outwardly extending projection 52 terminates, at each end of the lateral length 54, with a bump 53a, 53b. The side wall 38 of the lid reception section 34 of the base 12 further comprises a receiver 56 for the outwardly extending projection 52 of the lid 28 to releasably attach the lid 28 to the base 12. The receiver 56 has a lateral length 58 that is equal to or longer than the lateral length 54 of the outwardly extending projection 52 of the lid 28.

The receiver 56 of the side wall 38 of the lid reception section 34 of the base 12 further comprises a first overhang 60 and a second overhang 62. The first overhang 60 extends over the bump 53a of the outwardly extending projection 52 of the side wall 42 of the lid 28 when the lid 28 is in the first locked state 48 but not when the lid 28 is in the unlocked state 46 or the second locked state 50. As the lid 28 is moved to the first locked state 48, the first overhang 60 contacts and then snaps over the bump 53a of the outwardly extending projection 52 of the side wall 42 of the lid 28. The bump 53a and the first overhang 60 then cooperate to prevent the lid 28 from sliding back away from the first locked state 48 without the application of an intentional force.

The second overhang 62 extends over the bump 53b of the outwardly extending projection 52 of the side wall 42 of the lid 28 when the lid 28 is in the second locked state 50 but not when the lid 28 is in the unlocked state 46 or the first locked state 48. As the lid 28 is moved to the second locked state 50, the second overhang 62 contacts and then snaps over the bump 53b of the outwardly extending projection 52 of the side wall 42 of the lid 28. The bump 53b and the second overhang 62 then cooperate to prevent the lid 28 from sliding back away from the second locked state 50 without the application of an intentional force.

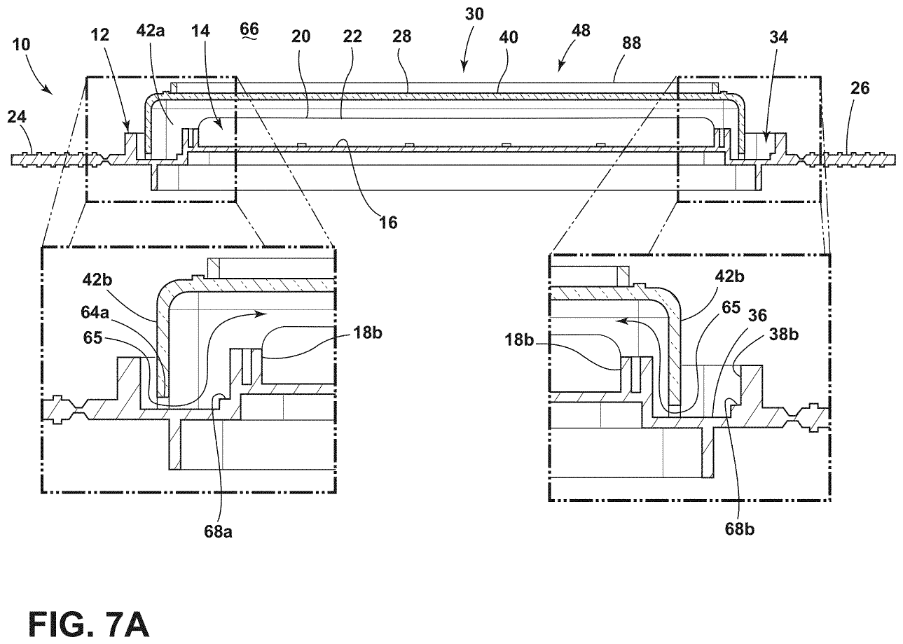

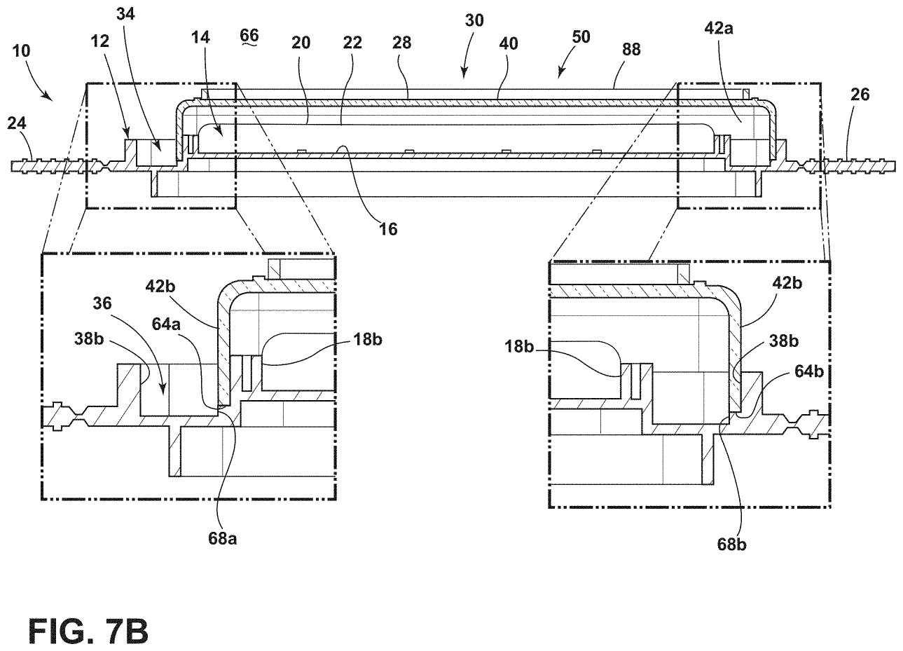

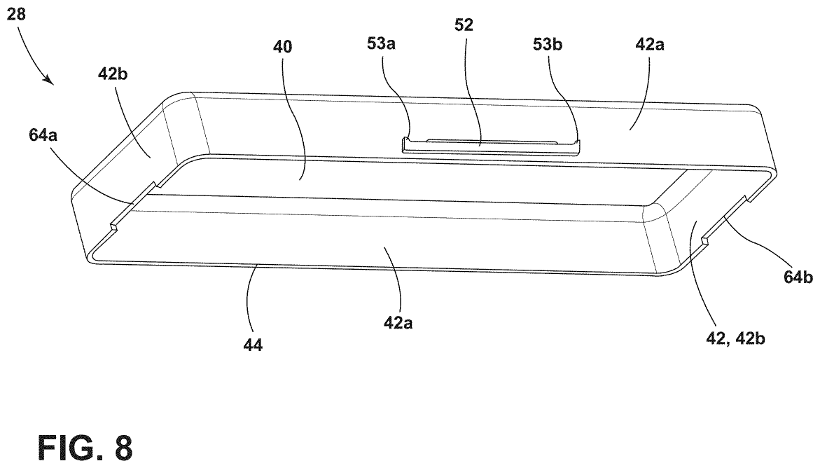

Referring now additionally to FIGS. 7A, 7B, and 8, the side wall 42 of the lid 28 includes a slot 64a, wherein when the lid 28 is in the first locked state 48 (see FIG. 7A), air 65 is able to flow from an environment exterior 66 to the microorganism collection device 10, through the slot 64a of the side wall 42 and to the culture medium 20. In embodiments, one of the pair of opposing side walls 42b of the lid 28 includes the slot 64a, and the other of the pair of opposing side walls 42b of the lid 28 includes a slot 64b. When the lid 28 is in the first locked state 48, air 65 is able to flow from the environment 66 exterior to the microorganism collection device 10, through both of the slots 64a, 64b, and to the culture medium 20.

The lid reception section 34 of the base 12 further includes a projection 68a that cooperates with the slot 64a when the lid 28 is in the second locked state 50 (see FIG. 7B) to reduce or prevent the air 65 from flowing from the environment exterior 66 to the microorganism collection device 10, through the slot 64a of the side wall 42, and to the culture media 20. In embodiments, the lid reception section 34 of the base 12 further includes a projection 68b that cooperates with the slot 64b when the lid 28 is in the second locked state 50 to reduce or prevent the air 65 from flowing from the environment exterior 66 to the microorganism collection device 10, through the slot 64b of the side wall 42, and to the culture medium 20. In embodiments, in the second locked state 50, the bottom edge 44 of the lid 28 sits upon the bottom 36 of the lid reception section 34 of the base 12 to prevent the air 65 from flowing between the bottom edge 44 of the lid 28 and the bottom 36 of the lid reception section 34 of the base 12 to the culture medium 20 from the environment exterior 66.

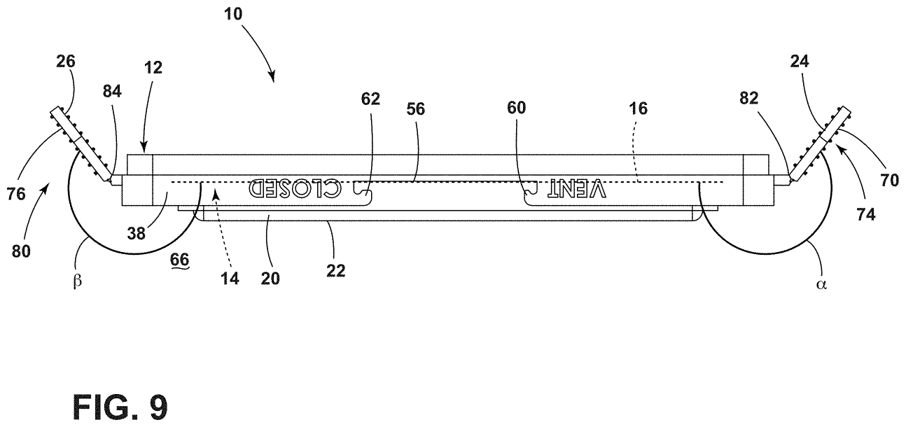

Referring now additionally to FIG. 9, the first tab 24 includes a top 70 that has an angular relationship α with the bottom 16 of the reservoir 14, a first position 72 (see, e.g., FIG. 1), and a second position 74 (see FIG. 9). The first tab 24 is movable to, from, and between the first position 72 and the second position 74. In the second position 74, the angular relationship α between the top 70 of the first tab 24 and the bottom 16 of the reservoir 14 is greater than in the first position 72. Likewise, the second tab 26 comprises a top 76 that has an angular relationship β with the bottom 16 of the reservoir 14, a first position 78 (see, e.g., FIG. 1), and a second position 80 (see FIG. 9). The second tab 26 is movable to, from, and between the first position 78 and the second position 80. In the second position 80, the angular relationship β between the top 76 of the second tab 26 and the bottom 16 of the reservoir 14 is greater than in the first position 78. The first tab 24 of the base 12 moves relative to the reservoir 14 about a hinge 82 when moving to, from, and between the first position 72 and the second position 74. The second tab 26 of the base 12 moves relative to the reservoir 14 about a hinge 84 when moving to, from, and between the first position 78 and the second position 80. The hinges 82, 84 can be a relative thinness in the material of which the base 12 is made (such as a molded plastic). The placement of the first tab 24 and the second tab 26 at opposite ends of the base 12, and the hinges 82, 84, allow a user to more easily take the base 12, maneuver the base 12 so that the culture medium 20 faces downward, and then press the top surface 22 of the culture medium 20 onto an object to collect a sample than dip slides and agar plates.

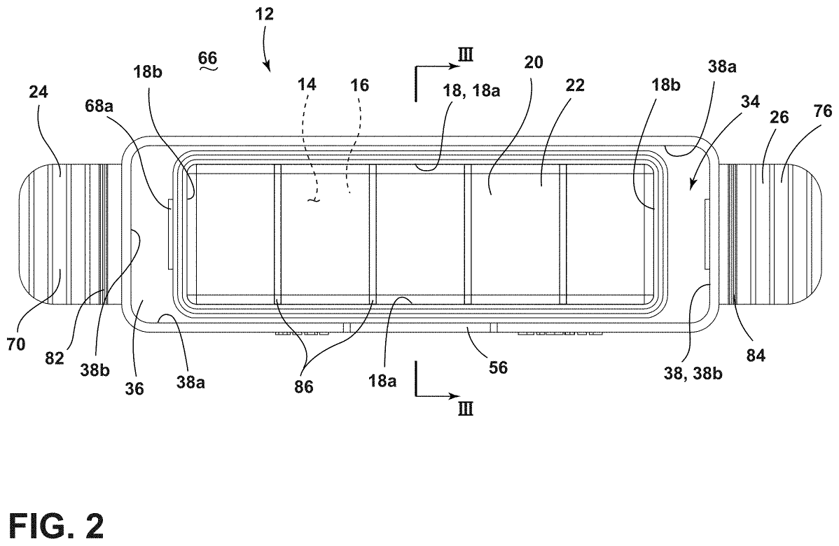

Referring back to FIG. 2, the reservoir 14 further comprises dividers 86 extending upward from the bottom 16 and disposed below the culture medium 20. The dividers 86 do not extend all the way up to the top surface 22 of the culture medium 20 so as to separate the culture medium 20 into different portions. However, the dividers 86 are visible through the culture medium 20.

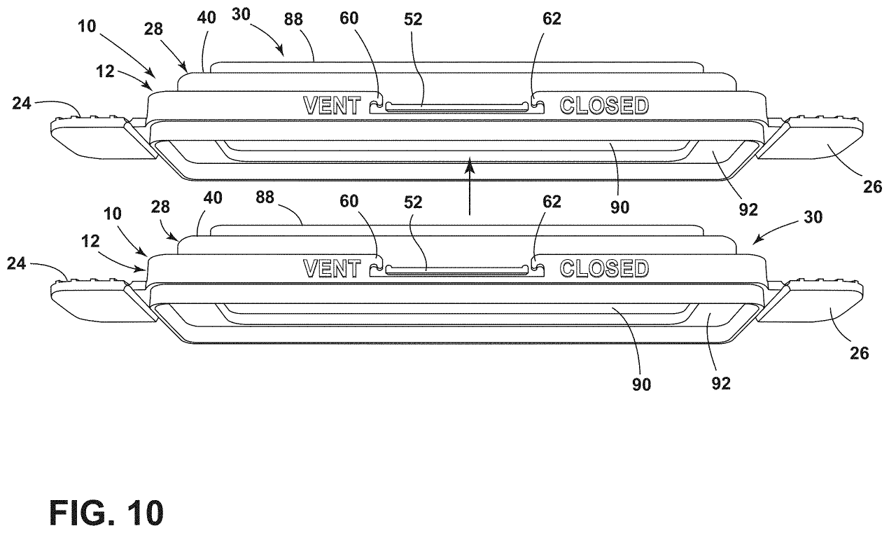

Referring now additionally to FIG. 10, in embodiments, the lid 28 further comprises a projection 88 projecting upward from the top 40. The base 12 further comprises a recessed portion 90 at an underside 92 of the base 12. The recessed portion 90 and the projection 88 are configured to mate with the recessed portion 90 of one microorganism collection device 10 (e.g., the top microorganism collection device 10 illustrated at FIG. 10) receiving the projection 88 of the lid 28 of another microorganism collection device 10 (e.g., the bottom microorganism collection device 10 illustrated at FIG. 10). The recessed portion 90, configured to receive the projection 88, allows multiple microorganism collection devices 10 to be stacked upon each other.

In embodiments, the microorganism collection device 10 further includes a machine readable indicia 94 (see FIG. 1). The machine readable indicia 94 can be a barcode (such as a linear barcode, a matrix code, and so on), a logo, a symbol, or any other indicia that a machine can identify. The machine can be a smartphone that captures an image of the machine readable indicia 94, and the smartphone can utilize application software to identify the machine readable indicia 94 and take designated actions after identifying the machine readable indicia 94. The machine readable indicia 94 can be disposed on the base 12 or on the lid 28, or both. The machine readable indicia 94 can be molded into the material from which the base 12 or lid 28 is made.

It is to be understood that variations and modifications can be made on the aforementioned structure without departing from the concepts of the present invention, and further it is to be understood that such concepts are intended to be covered by the following claims unless these claims by their language expressly state otherwise.

What is claimed is:

1. A microorganism collection device comprising:
a base, the base comprising (i) a reservoir comprising a bottom and a side wall extending up from the bottom, (ii) a culture medium within the reservoir, the culture medium disposed upon the bottom of the reservoir and comprising a top surface that is elevated from the bottom higher than the side wall of the reservoir, (iii) a first tab extending laterally outward relative to the reservoir; and
a lid comprising (i) an associated state where the lid is covering the culture medium and (ii) a dissociated state where the lid is separated from the base and not covering the culture medium.

2. The microorganism collection device of claim 1, wherein
the side wall of the reservoir comprises (i) a first pair of opposing side wall sections and (ii) a second pair of opposing side wall sections that are generally orthogonal to the first pair of opposing side wall sections.

3. The microorganism collection device of claim 1, wherein
the top surface of the culture medium is convex.

4. The microorganism collection device of claim 1, wherein
the reservoir further comprises a plurality of dividers extending upward from the bottom and disposed below the culture medium; and
the plurality of dividers are visible through the culture medium.

5. The microorganism collection device of claim 1, wherein
the base further comprises a lid reception section surrounding the reservoir, the lid reception section comprising a bottom and a side wall extending up from the bottom, the side wall of the lid reception section extending around the side wall of the reservoir.

6. The microorganism collection device of claim 5, wherein
the side wall of the lid reception section of the base comprises (i) a first pair of opposing side wall sections and (ii) a second pair of opposing side wall sections that are generally orthogonal to the first pair of opposing side wall sections.

7. The microorganism collection device of claim 1, wherein
the lid further comprises a top and a side wall extending down from the top; and
when the lid is in the associated state, the top of the lid is disposed over the culture medium, and the side wall of the lid extends around the side wall of the reservoir.

8. The microorganism collection device of claim 7, wherein
the lid further comprises a projection projecting upward from the top of the lid; and
the base further comprises a recessed portion at an underside of the base, the recessed portion of the microorganism collection device configured to receive the projection of the lid of another microorganism collection device to allow stacking of the microorganism collection devices.

9. The microorganism collection device of claim 5, wherein
the side wall of the lid comprises a bottom edge and, when the lid is in the associated state, the bottom edge of the lid faces the bottom of the lid reception section; and
when the lid is in the associated state, the side wall of the lid reception section of the base extends around the side wall of the lid, and the side wall of the lid is disposed between the side wall of the lid reception section and the side wall of the reservoir of the base.

10. The microorganism collection device of claim 1, wherein
the lid further comprises an unlocked state and a first locked state;
the lid is movable to, from, and between the unlocked state and the first locked state; and
when moving to, from, and between the unlocked state and the first locked state, the bottom edge of the lid slides laterally relative to the base.

11. The microorganism collection device of claim 10, wherein
the lid further comprises a second locked state and a slot;
when the lid is in the first locked state, air is able to flow from an environment exterior to the microorganism collection device, through the slot of the lid, and to the culture medium; and
the base further comprises a projection that cooperates with the slot when the lid is in the second locked state to reduce or prevent air from flowing from the environment exterior to the microorganism collection device, through the slot of the lid, and to the culture medium.

12. The microorganism collection device of claim 1, wherein
the lid further comprises an outwardly extending projection, the outwardly extending projection comprising a lateral length; and
the base further comprises a receiver for the outwardly extending projection of the lid to releasably attach the lid to the base, the receiver comprising a lateral length that is equal to or longer than the lateral length of the outwardly extending projection of the lid.

13. The microorganism collection device of claim 11, wherein
the lid further comprises an outwardly extending projection, the outwardly extending projection comprising a lateral length;
the base further comprises a receiver for the outwardly extending projection of the lid to releasably attach the lid to the base, the receiver comprising a lateral length that is equal to or longer than the lateral length of the outwardly extending projection of the lid;
the receiver of the base further comprises a first overhang and a second overhang;
the first overhang extends over the outwardly extending projection of the side wall of the lid when the lid is in the first locked state but not when the lid is in the unlocked state or the second locked state; and
the second overhang extends over the outwardly extending projection of the side wall of the lid when the lid is in the second locked state but not when the lid is in the unlocked state or the first locked state.

14. The microorganism collection device of claim 1, wherein
the first tab comprises a top that has an angular relationship with the bottom of the reservoir; and
the first tab is movable to, from, and between a first position, and a second position where the angular relationship between the top of the first tab and the bottom of the reservoir is greater than in the first position.

15. The microorganism collection device of claim 14, wherein
the first tab of the base moves relative to the reservoir about a hinge when moving to, from, and between the first position and the second position.

16. The microorganism collection device of claim 15 further comprising:
a second tab extending outward relative to the reservoir away from the first tab;
wherein, the second tab comprises a top that has an angular relationship with the bottom of the reservoir;
wherein, the second tab is movable to, from, and between a first position, and a second position where the angular relationship between the top of the second tab and the bottom of the reservoir is greater than in the first position; and
wherein, the second tab of the base moves relative to the reservoir about a hinge when moving to, from, and between the first position and the second position.

17. The microorganism collection device of claim 1 further comprising:
a machine readable indicia.

18. A microorganism collection device comprising:
a base, the base comprising (i) a reservoir comprising a bottom and a side wall extending up from the bottom, and (ii) a culture medium within the reservoir, the culture medium disposed upon the bottom of the reservoir and comprising a top surface that is elevated from the bottom higher than the side wall of the reservoir; and
a lid comprising (i) a top, (ii) a side wall extending down from the top, the side wall comprising a bottom edge, (iii) an associated state where the lid is coupled to the base and the top of the lid is disposed over the culture medium, (iv) a dissociated state where the lid is not coupled to the base and not covering the culture medium, (v) an unlocked state, (vi) a first locked state, and (vii) a second locked state;
wherein, the lid is movable to, from, and between the unlocked state, the first locked state, and the second locked state;
wherein, when moving to, from, and between the unlocked state to the first locked state, the bottom edge of the lid slides laterally relative to the base in a first direction; and
wherein, when moving to, from, and between the unlocked state to the second locked state, the bottom edge of the lid slides laterally relative to the base in a second direction, the first direction and the second direction being opposites.

19. The microorganism collection device of claim 18, wherein
the side wall of the lid further comprises an outwardly extending projection, the outwardly extending projection comprising a lateral length;
the base further comprises a lid reception section around the reservoir, the lid reception section comprising a bottom and a side wall extending up from the bottom, the side wall of the lid reception section extending around the side wall of the reservoir;
the side wall of the lid reception section of the base comprises a receiver configured to cooperate with the outwardly extending projection of the lid to attach the lid to the base, the receiver comprising (i) a lateral length that is longer than the lateral length of the outwardly extending projection of the lid, (ii) a first overhang, and (iii) a second overhang;
the first overhang extends over the outwardly extending projection of the side wall of the lid when the lid is in the first locked state but not when the lid is in the unlocked state or the second locked state; and
the second overhang extends over the outwardly extending projection of the side wall of the lid when the lid is in the second locked state but not when the lid is in the unlocked state or the first locked state.

20. A microorganism collection device comprising:
a base, the base comprising (i) a reservoir comprising a bottom and a side wall extending up from the bottom, and (ii) a culture medium within the reservoir, the culture medium disposed upon the bottom of the reservoir and comprising a top surface that is elevated from the bottom higher than the side wall of the reservoir; and
a lid comprising (i) a top, (ii) a side wall extending down from the top, the side wall of the lid comprising a slot, (iii) an associated state where the lid is coupled to the base and the top of the lid is disposed over the culture medium, (iv) a dissociated state where the lid is not coupled to the base and not covering the culture medium, (v) an unlocked state, (vi) a first locked state in which air is able to flow from an environment exterior to the microorganism collection device, through the slot of the side wall, and to the culture medium, and (vii) a second locked state in which the slot cooperates with a projection of the base to reduce or prevent air from flowing from the environment exterior to the microorganism collection device, through the slot of the side wall, and to the culture medium;
wherein, the lid is movable to, from, and between the unlocked state, the first locked state, and the second locked state; and
wherein, the side wall of the reservoir comprises (i) a first pair of opposing side wall sections that are planar and (ii) a second pair of opposing side wall sections that are planar and orthogonal to the first pair of opposing side wall sections.
